(19) United States
(12) Patent Application Publication
(10) Pub. No.: US 2009/0054752 A1
(43) Pub. Date: Feb. 26, 2009

(54) METHOD AND APPARATUS FOR PHOTOPLETHYSMOGRAPHIC SENSING

(75) Inventors: Krishna Jonnalagadda, Algonquin, IL (US); Di-An Hong, Inverness, IL (US); Xun Luo, Cicero, IL (US); Francesca Schuler, DesPlaines, IL (US); Andrew Skipor, West Chicago, IL (US)

Correspondence Address: INGRASSIA FISHER & LORENZ, P.C. (MOT), 7010 E. Cochise Road, SCOTTSDALE, AZ, 85253, US

(73) Assignee: MOTOROLA, INC., Schaumburg, IL (US)

(21) Appl. No.: 11/843,187

(22) Filed: Aug. 22, 2007

(51) Int. Cl. A61B 5/02

(52) U.S. Cl. 600/324

(57) ABSTRACT

A photoplethysmographic sensing system for determining a user's pulse rate includes a light emitting device (100, 201, 310, 500) including a first plurality of light emitting particles (108, 208, 317) having a first diameter and emitting light having a first wavelength. A detector (118, 218, 500) is positioned to receive light emitted from the plurality of light emitting particles (108, 208, 317) and a processing device (500) determines the pulse rate. The light emitting device (100, 201, 310, 500) and the detector (118, 218, 500) are disposed on a flexible polymeric material (102, 202, 334). The light emitting device (100, 201, 310, 500) may include a second plurality of light emitting particles (108, 208, 317) having a second diameter and emitting light having a second wavelength, wherein the processing device (500) determines the user's blood oxygen level. The light emitting particles (108, 208, 317) may comprise one of quantum dots, electroluminescent particles, or organic particles.

### FIELD

The present invention generally relates to photoplethysmographic sensors and more particularly to a photoplethysmographic sensor for active lifestyles.

### BACKGROUND

Photoplethysmography (PPG) is the process of applying a light source, e.g., a light emitting diode (LED), and light sensor, e.g., a photodiode, to an appendage, such as a finger, toe, ear, or wrist, and measuring the reflected light. At each contraction of the heart, blood is forced through the peripheral vessels producing engorgement of the vessels under the light source, thereby modifying the amount of light provided to the photo sensor. Since vasomotor activity is controlled by the sympathetic nervous system, the Blood Volume Pulse (BVP) measurements can display changes in sympathetic arousal. An increase in BVP amplitude indicates decreased sympathetic arousal and greater blood flow to the peripheral vessels.

It is desired that PPG sensors and measurements made with a PPG sensor include an accurate co-location of the light source and sensor, conformity of the sensor to body contours, providing of adequate light to the sensor, proximity of light sources for SPO₂ (oxygen saturation) measurement, and motion tolerance (accuracy in view of body movement).

Accordingly, it is desirable to provide a PPG sensing system to detect a user's pulse rate and oxygen saturation. Furthermore, other desirable features and characteristics of the present invention will become apparent from the subsequent detailed description and the appended claims, taken in conjunction with the accompanying drawings and this background.

### BRIEF DESCRIPTION OF THE DRAWINGS

Embodiments of the present invention will hereinafter be described in conjunction with the following drawing figures, wherein like numerals denote like elements, and

FIG. 1 is a partial schematic cross section of a first exemplary embodiment;

FIG. 2 is partial schematic cross section of a second exemplary embodiment;

FIG. 3 is partial schematic cross section of a third exemplary embodiment;

FIG. 4 is a partial schematic cross section of a fourth exemplary embodiment;

FIG. 5 is a graph of absorbance versus wavelength for non-oxygenated (Hb) and oxyhemoglobin (HbO₂) of blood;

FIG. 6 is a block diagram of a signal processing device for heart rate computing in accordance with exemplary aspects of the disclosure; and

FIG. 7 is a block diagram of a fifth exemplary embodiment.

### DETAILED DESCRIPTION

Desirable features and characteristics of the present invention will become apparent from the subsequent detailed description and the appended claims, taken in conjunction with the accompanying drawings and this background.

An exemplary embodiment of a photoplethysmographic (PPG) sensing system includes a free standing quantum dot (FSQD) enabled emission source that provides at least one wavelength of light and at least one photodiode receiver. The FSQDs are dispersed in a polymer or other flexible material and are driven by a photonic or electronic source. One wavelength is used to measure the heart rate, while two wavelengths (when an optional second wavelength is provided) may be used to measure the blood oxygen level and to increase the accuracy of the pulse signal. The flexible material allows the PPG sensor to follow body contours and potentially increases the light provided to the photodiode. The PPG sensor therefore requires less contact pressure to obtain sufficient data at the photodiode. The PPG sensor is also tolerant of local loss of contact between the sections of the light source and the skin since other sections of the distributed light source are in contact. The data may be transmitted continuously or periodically to a remote control center for additional monitoring and analysis. The PPG sensing system may also include a one or multi-dimensional accelerometer to provide data regarding the user's body motion for noise cancellation. Advanced data processing algorithms such as adaptive windowing, non-linear modeling and Q-filter related approaches may be used to provide valid heart-rate measurements.

The exemplary embodiment described herein provides a strong PPG signal due to a larger lighted area and quantum dot efficiency, conforms to body contours which broadens locations where an accurate PPG signal can be obtained, provides an optimal relative location of the FSQD light source and photodiode, reduces contact pressure on the skin, and provides multiple accurate wavelengths from the same source. And since the preferred emitted wavelengths are red and infrared, the photons can be activated by blue light without using environmentally unfriendly ultraviolet light. In addition to the FSQD light source and photodiode, the PPG sensing system may be embodied within or may function in conjunction with a cellular phone, digital music player, earwear or eyewear for use in athlete training, elder health care, and fitness activities, for example.

Free standing quantum dots (FSQDs) are semiconductor nanocrystallites whose radii are smaller than the bulk exciton Bohr radius and constitute a class of materials intermediate between molecular and bulk forms of matter. FSQDs are known for the unique properties that they possess as a result of both their small size and their high surface area to volume ratio. For example, FSQDs typically have larger absorption cross-sections than comparable organic dyes, higher quantum yields, better chemical and photo-chemical stability, narrower and more symmetric emission spectra, and a larger Stokes shift. Furthermore, the absorption and emission properties vary with the particle size and can be systematically tailored. It has been found that a Cadmium Selenium (CdSe) quantum dot, for example, can emit light in any monochromatic, visible color, where the particular color characteristic of that dot is dependent on the size of the quantum dot, (i.e., size tunable band gap).

FSQDs are easily incorporated (solubalized or dispersed) into or onto other materials such as polymers and polymer composites because solution processing of inorganic nanocrystals is made possible by a capping layer of organic capping groups on the surface of the FSQDs. This capping layer may be tailored to control solubility, external chemistry, and particle spacing. FSQDs are highly soluble and have little degradation over time.

Free standing quantum dots (FSQDs) are semiconductors including, for example, periodic groups of II-VI, III-V, or IV-VI materials, for example, CdS, CdSe, CdTe, ZnS, ZnSe, ZnTe, GaAs, GaP, GaAs, GaSb, HgS, HgSe, HgTe, InAs, InP, InSb, AlAs, AlP, AlSb. Alternative FSQDs materials that may be used include but are not limited to tertiary microcrystals such as InGaP, which emits in the yellow to red wavelengths (depending on the size) and ZnSeTe, ZnCdS, ZnCdSe, and CdSeS which emits from blue to green wavelengths. Multi-core FSQD structures are also possible such as ZnSe/ZnXS/ZnS, where the innermost core is made of ZnSe, followed by a second core layer of ZnXS, and completed by an external shell made of ZnS, where X represents Strontium (Sr), Tellurium (Te), Silver (Ag), Copper (Cu) or Manganese (Mn).

FSQDs range in size from 2-10 nanometers in diameter (approximately 10²-10⁷ total number of atoms). At these scales, FSQDs have size-tunable band gaps, in other words there spectral emission depends upon size. Whereas, at the bulk scale, emission depends solely on the composition of matter. Other advantages of FSQDs include high photoluminescence quantum efficiencies, good thermal and photo-stability, narrow emission line widths (atom-like spectral emission), and compatibility with solution processing. FSQDs are manufactured conventionally by using colloidal solution chemistry.

FSQDs may be synthesized with a wider band gap outer shell, comprising for example ZnO, ZnS, ZnSe, ZnTe, CdO, CdS, CdSe, CdTe, MgS, MgSe, GaAs, GaN, GaP, GaAs, GaSb, HgO, HgS, HgSe, HgTe, InAs, InN, InP, InSb, AlAs, AlN, AlP, AlSb. The shell surrounds the core FSQDs and results in a significant increase in the quantum yield. Capping the FSQDs with a shell reduces non-radiative recombination and results in brighter emission. The surface of FSQDs without a shell has both free electrons in addition to crystal defects. Both of these characteristics tend to reduce quantum yield by allowing for non-radiative electron energy transitions at the surface. The addition of a shell reduces the opportunities for these non-radiative transitions by giving conduction band electrons an increased probability of directly relaxing to the valence band. The shell also neutralizes the effects of many types of surface defects. The FSQDs are more thermally stable than organic phosphors since UV light will not chemically breakdown FSQDs. The exterior shell can also serve as an anchor point for chemical bonds that can be used to modify and functionalize the surface.

Due to their small size, typically on the order of 10 nanometers or smaller, the FSQDs have larger band gaps relative to a bulk material. It is noted that the smaller the FSQDs, the higher the band gap. Therefore, when impacted by a photon (emissive electron-hole pair recombination), the smaller the diameter of the FSQDs, the shorter the wavelength of light will be released. Discontinuities and crystal defects on the surface of the FSQD result in non-radiative recombination of the electron-hole pairs that lead to reduced or completely quenched emission of the FSQD. An overcoating shell (example ZnS) having, e.g., a thickness of up to 5 monolayers and higher band gap compared to the core's band gap is optionally provided around the FSQDs core to reduce the surface defects and prevent this lower emission efficiency. The band gap of the shell material should be larger than that of the FSQDs to maintain the energy level of the FSQDs. Capping ligands (molecules) on the outer surface of the shell allow the FSQDs to remain in the colloidal suspension while being grown to the desired size. The FSQDs may then be placed by a printing process, for example. Additionally, a light source is disposed to selectively provide photons to strike the FSQDs, thereby causing the FSQDs to emit a photon at a frequency comprising the specific color as determined by the size tunable band gap of the FSQDs. Alternatively, a voltage may be applied across the FSQDs, thereby causing the FSQDs to emit photons.

The exemplary embodiments described herein may be fabricated using known lithographic processes as follows. The fabrication of integrated circuits, microelectronic devices, micro electro mechanical devices, microfluidic devices, and photonic devices, involves the creation of several layers of materials that interact in some fashion. One or more of these layers may be patterned so various regions of the layer have different electrical or other characteristics, which may be interconnected within the layer or to other layers to create electrical components and circuits. These regions may be created by selectively introducing or removing various materials. The patterns that define such regions are often created by lithographic processes. For example, a layer of photoresist material is applied onto a layer overlying a wafer substrate. A photomask (containing clear and opaque areas) is used to selectively expose this photoresist material by a form of radiation, such as ultraviolet light, electrons, or x-rays. Either the photoresist material exposed to the radiation, or that not exposed to the radiation, is removed by the application of a developer. An etch may then be applied to the layer not protected by the remaining resist, and when the resist is removed, the layer overlying the substrate is patterned. Alternatively, an additive process could also be used, e.g., building a structure using the photoresist as a template.

Though various lithography processes, e.g., photolithography, electron beam lithography, and various printing processes including imprint lithography ink jet printing, may be used to fabricate the light emitting device 200, a printing process is preferred. In the printing process, the FSQD ink in liquid form is printed in desired locations on the substrate. Ink compositions typically comprise four elements: 1) functional element, 2) binder, 3) solvent, and 4) additive. Graphic arts inks and functional inks are differentiated by the nature of the functional element, i.e. the emissive quantum dot. The binder, solvent and additives, together, are commonly referred to as the carrier which is formulated for a specific printing technology e.g. tailored rheology. The function of the carrier is the same for graphic arts and printed electronics: dispersion of functional elements, viscosity and surface tension modification, etc. One skilled in the art will appreciate that an expanded color range can be obtained by using more than three quantum dot inks, with each ink having a different mean quantum dot size. A variety of printing techniques, for example, Flexo, Gravure, Screen, inkjet may be used. The Halftone method, for example, allows the full color range to be realized in actual printing.

One manufacturing process providing a PPG sensor on a flexible structure includes laminating a non-patterned multilayer film, e.g., a transparent conductive film, an electroluminescent layer, or a dielectric layer, to a patterned substrate, e.g., a patterned conductor for pixel or printed pixel driving circuits. Another manufacturing process includes printing an electrode to define a pixel on a polyethylene terephthalate substrate, printing an electroluminescent material on the pixel and printing or covering with a dielectric material, and laminating with a transparent conductor.

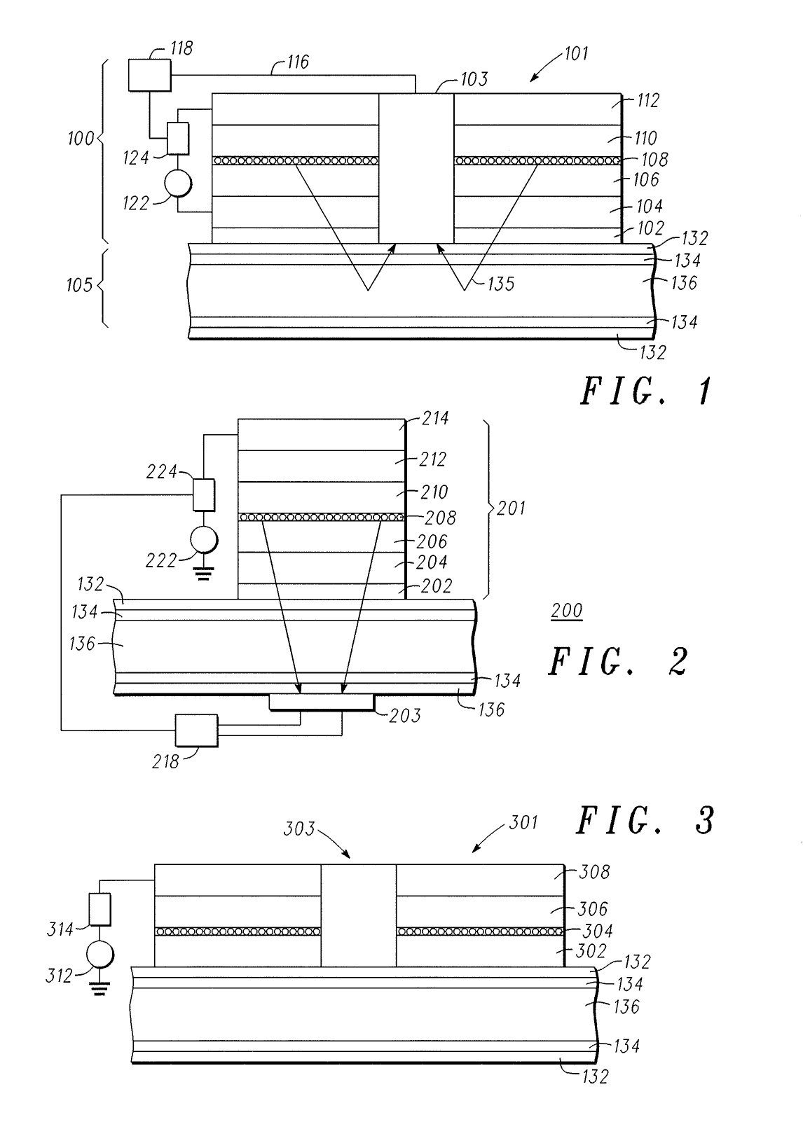

Referring to FIG. 1, a cross sectional view of a photoplethysmographic (PPG) sensing system 100, positioned on, e.g., a human appendage, includes a light emitting device 101 and a photodiode 103. The light emitting device 101 includes a first electrode 104 formed on a substrate 102. The substrate is formed of a transparent, flexible, thin material, for example a flexible polymer such as polyethylene terephthalate (PET) or polyethylene naphthalate (PEN). The first electrode 104 (anode) comprises, for example, a transparent material, preferably indium tin oxide. A hole transport layer 106 of, for example, indium tin oxide (ITO), poly-3,4-ethylenedioxthiophene (PEDOT), or N,N0-diphenyl-N,N0-bis(3-methylphenyl)-(1,1 0-biphenyl)-4,4 0-diamine (TPD) is formed on the first electrode 104. The hole transport layer 106 may alternatively comprise an electron blocking layer. A layer 108 of a plurality of FSQDs, including FSQDs of at least one size as discussed hereinafter, is formed on the hole transport layer 106. Note that the hole and transport layers could also be made of inorganic materials. An electron injection layer 110 and a second electrode 112 are then formed over the layer 108. The electron injection layer 110 may be either organic or inorganic and comprise, e.g., tris-(8-hydroxyquinoline)aluminium or 3-(4-Biphenylyl)-4-phenyl-5-tert-butylphenyl-1,2,4-triazole (TAZ). The second electrode 112 (cathode) may, in this exemplary embodiment, be an opaque electron source material including, for example, magnesium and silver. A reflective surface (not shown) may be disposed between the electron injection layer 110 and the second electrode 112 to increase the directionality of the photons.

The photodiode 103 is positioned adjacent to the substrate 102. In this exemplary embodiment, the photodiode 103 is surrounded by the layers 102, 104, 106, 108, 110, 112, forming the light emitting device 101. An output 116 of the photodiode 101 is coupled to electronics 118. A voltage source 122 is coupled between electrodes 104 and 112 by a switch 124. The photodiode 103 may be any photodiode known in the industry that is sensitive to the wavelength emitted by the light emitting device 101.

The photoplethysmographic (PPG) sensing system 100 is placed on an appendage 105 of a user and is represented schematically in FIG. 1 as a layer of fat 134 between a layer of skin 132 and a blood vessel 134. When data regarding a pulse rate (subsequently discussed in more detail) is desired from the photoplethysmographic (PPG) sensing system 100, a signal 142 from electronics 118 activates the switch 124, thereby applying a voltage across the electrodes 104, 112. In response to this voltage, an electron in each of the FSQDs is excited to a higher level. When the electron falls back to its ground state, a photon is emitted having a wavelength determined by the diameter of the FSQD. Though some of this light 136 may be reflected by the skin 132 and the fat 134, much is reflected by the blood within the vessel 136 back to the photodiode 103.

Referring to FIG. 2 and in accordance with a second exemplary embodiment, a cross sectional view of a photoplethysmographic (PPG) sensing system 200 includes a light emitting device 201 and a photodiode 203. The light emitting device 201 includes a substrate 202, an electrode 204, a hole transparent layer 106, a layer 208 of FSQDs, an electron injection layer 210, and another electrode 212 as described with the previous embodiment of FIG. 1; however, the electrode 212 will be transparent in this exemplary embodiment. The light emitting device further comprises a light source 214 deposited on the electrode 212. The light source 214 is coupled to a voltage source 216 through switch 218 for selectively activating the light source 214. It is understood that the light source 214 may be positioned in any location wherein its output may be applied to the FSQDs, and may comprises any frequency below that provided as output from the FSQDs, but preferably comprises blue light, though other wavelengths could be used, including ultraviolet (UV).

In operation, when the layer 208 of the plurality of FSQDs is impacted with light having a wavelength shorter that which would be emitted by the FSQDs, an electron in each of the FSQDs so impacted is excited to a higher level. When the electron falls back to its ground state, a photon is emitted having a wavelength determined by the size of the FSQD. The level of photon emission from the FSQDs may be controlled by varying the voltage potential of the voltage source 216 by the switch 218.

Referring to FIG. 3 and in accordance with a third exemplary embodiment, a cross sectional view of a photoplethysmographic (PPG) sensing system 300 includes a light emitting device 301 and a photodiode 303. The light emitting device 301 includes a substrate 302, a layer 304 of FSQDs, and an optional substrate 306. The light emitting device 300 further comprises a light source 308 positioned either on the layer 304 or the optional substrate 306. The light source 308 is coupled to a voltage source 312 through switch 314 for selectively activating the light source 308. It is understood that the light source 308 may be positioned in any location wherein its output may be applied to the FSQDs, and may comprises any frequency below that provided as output from the FSQDs, but preferably comprises blue light, though other wavelengths could be used, including ultraviolet (UV). The light source 308 preferably is an electroluminescent (EL) lamp, which is basically a luminescent capacitor. By applying alternating voltage, phosphor particles that are dispersed in dielectric get excited and emit light. An EL lamp is a solid state, low power, uniform area light source with a thin profile. By applying alternating voltage to the electrodes, phosphor particles that are dispersed in dielectric get excited and emit light through a transparent electrode.

In operation, when the layer 304 of the plurality of FSQDs is impacted with light from the light source 308 having a wavelength shorter that which would be emitted by the FSQDs, an electron in each of the FSQDs so impacted is excited to a higher level. When the electron falls back to its ground state, a photon is emitted having a wavelength determined by the size of the FSQD. The level of photon emission from the FSQDs may be controlled by varying the voltage potential of the voltage source 312 by the switch 314.

A fourth exemplary embodiment (FIG. 4) includes an electroluminescent lamp as a light source. Electroluminescent (EL) lamps are basically luminescent capacitors. By applying alternating voltage to the electrodes, phosphor particles that are dispersed in dielectric get excited and emit light. An Electroluminescent (EL) lamp is a solid state, low power, uniform area light source with a thin profile. It is, basically, a flat luminescent capacitor. By applying alternating voltage to the electrodes, phosphor particles that are dispersed in dielectric get excited and emit light through a transparent electrode. EL is an effective thin lighting solution that is used to backlight applications that need to be visible in dark conditions. It is frequently used in monochrome displays and keypads of portable handheld products such as cell phones (handsets), PDAs, MP3/CD players, pagers, cordless phones, remote controls, medical devices, and timepieces (clocks/wristwatches). It is also used in many automotive interior applications such as instrument clusters, radios, climate controls and switch assemblies.

EL lamps offer significant advantages over point light sources such as discrete light emitting diodes (LEDs), which are not as efficient. For example, the high LED count that is required to evenly light large liquid crystal displays (LCDs) consumes more current than an alternative EL backlight system. In addition, LED solutions normally require a complex light guide design to distribute the light more uniformly across the viewing area of a display. This combination of LEDs and light guide is generally three to four times thicker than an EL lamp solution.

An electroluminescent display device contains an electroluminescent phosphor sandwiched between a pair of electrodes. Referring now to FIG. 4, the electroluminescent device 410 includes a substrate 412 that has a bottom electrode 414 situated thereon. A layer of electroluminescent material 416 including phosphor particles 417, and a dielectric layer 418 are situated between the bottom electrode 414 and a top electrode 420. A source of alternating voltage 424 is coupled to the top and bottom electrodes to energize the electroluminescent material. An optically transmissive insulating or dielectric layer 422 is disposed over the top electrode 420.

A photodiode 424 is positioned adjacent to the the photodiode 103, and more particularly in this embodiment is surrounded by the layers 412, 414, 416, 418, 420, 422. The photodiode 424 may be any photodiode known in the industry that is sensitive to the wavelength emitted by the light emitting device 410.

The photoplethysmographic (PPG) sensing system 410 is placed on an appendage 430 of a user and is represented schematically in FIG. 4 as a layer of fat 432 between a layer of skin 434 and a blood vessel 436. When data regarding a pulse rate (subsequently discussed in more detail) is desired from the photoplethysmographic (PPG) sensing system 410, a voltage is applied across the electrodes 414, 420. In response to this voltage, an electron in each of the phosphors is excited to a higher level. When the electron falls back to its ground state, a photon is emitted having a wavelength determined by the phosphor selected. Though some of this light 440 may be reflected by the skin 434 and the fat 432, much is reflected by the blood within the vessel 436 back to the photodiode 424.

Approximately fifteen percent of blood by weight is hemoglobin inside the red blood cells. The total Hb mostly (about 99%) comprises reduced or non-oxygenated (Hb) and oxyhemoglobin (HbO₂). Transmittance of light through an absorbing medium is defined by T=I/I₀, where I is the transmitted intensity and I₀ is the incident intensity. Absorbance is given by A=−log₁₀ T. Absorbance may further be expressed as A=log (I₀/I)=(In10)cεL (Beer's Law), where ε is the molar absorptivity (in cm⁻¹ M⁻¹), L is the path length, and c is the molar concentration.

As arterial pulsations fill the capillaries, the changes in volume of the blood vessels modify the absorption, reflection, and scattering of the light. The amount of HbO changes also, resulting in additional modulation. SpO₂ is a measurement of the amount of oxygen attached to the haemoglobin cell in the circulatory system, or restated, is the amount of oxygen (saturation) being carried by the red blood cell in the blood. SpO₂ is given in as a percentage of total Hb, with normal for a human being around 96%. Generally, the magnitude for SpO₂ goes up and down according to how well a person is respiring (breathing) and how well the blood is being pumped around the body.

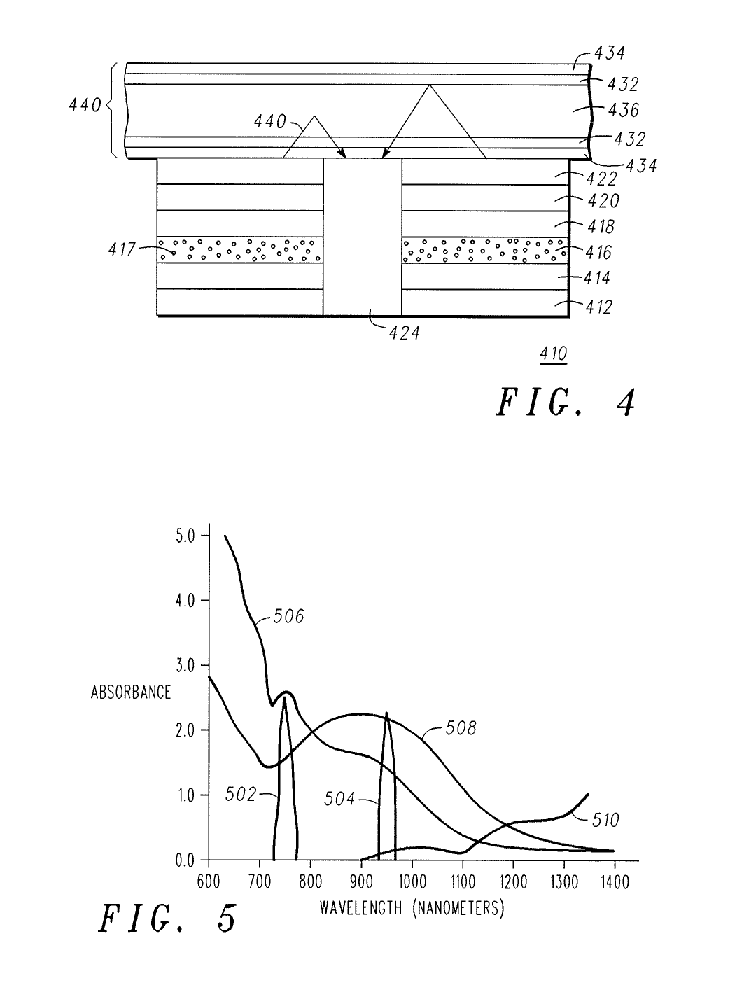

While a single wavelength may be used to determine the pulse rate, two wavelengths may determine the ratio of HbO₂ (oxygen levels). FIG. 5 is a graph showing quantum dots 502, 504 having a wavelength of 750 and 950 nanometers, respectively. Trace 406 represents the non-oxygenated (Hb), trace 408 represents the oxyhemoglobin (HbO₂), and trace 510 represents water. By comparing the light absorption at the two wavelengths, the blood oxygen level can be calculated. Since both the light sources are co-located and geometrically identical, there is no error at the detector due to the distance and condition of the light sources. For example, if one section of the light source has detached from the skin, both light sources are “equally” detached. With 2 LEDs, one may be attached while the other is detached leading to potentially lower signal to noise ratio.

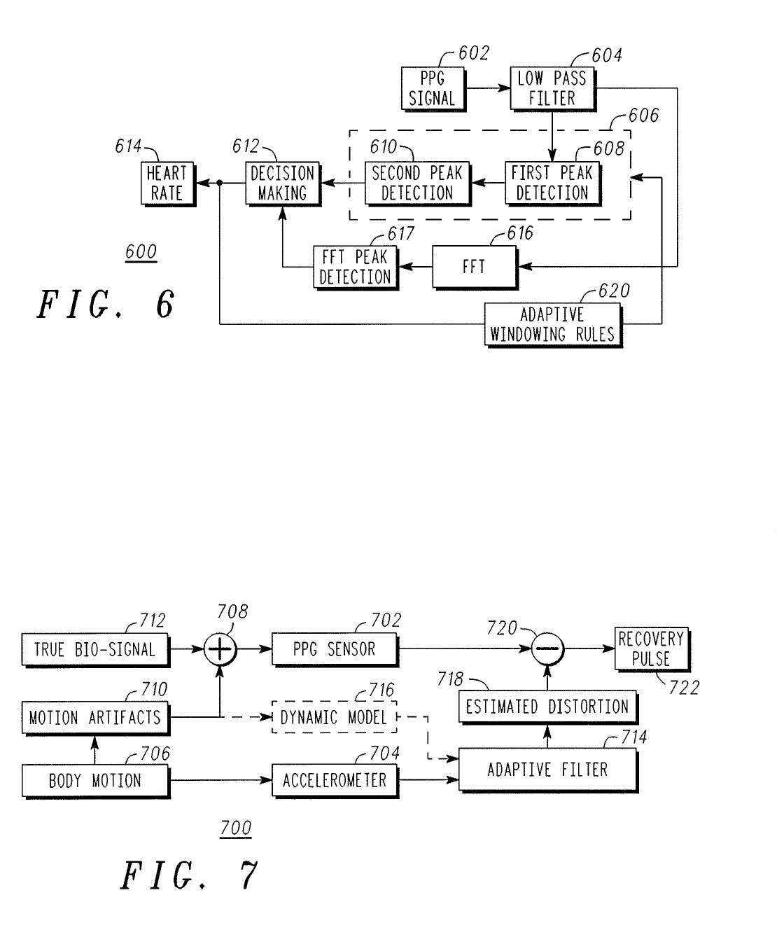

Referring now to FIG. 6, a block diagram 600 illustrates a signal processing device for determining heart rate without active noise cancellation. A PPG signal 602 is processed by a low pass filter 604. The resultant signal is then sent to two processing devices. A first processing device comprises a detection algorithm 606 examining a first peak detection 608 and a second peak detection 610. The output of the detection algorithm is then processed by a decision making algorithm 612 where a heart rate determination is made and subsequently output 614.

The second processing device encountered by the output of the low pass filter 604 is a Fast Fourier Transform 616 followed by a Fast Fourier Transform peak detection 618. The output of the Fast Fourier Transform peak detection 618 is processed by the decision making algorithm 612 where the heart rate determination is made and then provided as an output 614.

In all cases, the heart rate determination output from the decision making algorithm 612 is fed back to the detection algorithm 606, but is first processed by an adaptive windowing process 620. The adaptive windowing process 620 dynamically alters the data window size depending on previous heart rate measurements and determinations based on data quality.

FIG. 7 is a block diagram of a third exemplary embodiment of a photoplethysmo-graphic (PPG) sensing system 700, for providing active cancellation including a PPG sensor 702 and an accelerometer 704. Active noise cancellation is conducted by comparing the signals from the PPG sensor and the accelerometer and subtracting the extraneous noise. Motion 706 of the appendage, or body part, to which the PPG sensor 702 is attached, is sensed by the accelerometer 704 and is ADDED 708 as motion artifacts 710 with a true bio-signal 712, thereby impacting the PPG sensor 702. An adaptive filter 714, in response to the accelerometer 704 and a dynamic model 716, provides an estimated distortion 718 to be SUBTRACTED from the output of the PPG sensor 701 for providing an accurate recovered pulse 722.

The active noise cancellation is performed by obtaining a signature of the noise profile from the accelerometer 704 and dynamically subtracting 720 the recorded noise 706 (motion, or vibration) from the PPG sensor 702, which is subjected to the same noise 706. The digital signals are preprocessed using an amplifier and a spectrum filter. Variations of the LMS and RLS algorithms as well as other types of algorithms can be used to process the data.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims.

1. A photoplethysmographic sensing system for determining a user's pulse rate, comprising:
a light emitting device including a first plurality of light emitting particles having a first diameter;
a detector for receiving light emitted from the plurality of light emitting particles; and
a processing device coupled to the detector for determining the pulse rate.

2. The photoplethysmographic sensing system of claim 1 wherein the light emitting device includes a second plurality of light emitting particles, wherein the processing device further determines the user's blood oxygen level.

3. The photoplethysmographic sensing system of claim 1 wherein the first plurality of light emitting particles comprise a first plurality of quantum dots.

4. The photoplethysmographic sensing system of claim 2 wherein the second plurality of light emitting particles comprise a second plurality of quantum dots.

5. The photoplethysmographic sensing system of claim 1 wherein the first plurality of light emitting particles comprise electroluminescent particles.

6. The photoplethysmographic sensing system of claim 1 wherein the first plurality of light emitting particles comprise organic light emitting particles.

7. The photoplethysmographic sensing system of claim 1 wherein the light emitting device and the detector are disposed on a flexible polymeric substrate.

8. The photoplethysmographic sensing system of claim 1 further comprising one of a light source and a voltage source for activating the light emitting device.

9. The photoplethysmographic sensing system of claim 1 further comprising an accelerometer coupled to the light emitting device for compensating for motion imparted to the light emitting device.

10. The photoplethysmographic sensing system of claim 1 wherein the detector is disposed adjacent to the light emitting device.

11. A photoplethysmographic sensing system comprising:
a flexible material;
a light emitting device disposed on the flexible material, comprising:
a cathode;
a electron transparent layer formed over the cathode;
a first plurality of light emitting particles, each having a first diameter, disposed over the electron transport layer;
a hole transport layer formed over the plurality of light emitting particles; and
an anode formed over the hole transport layer;
a detector for receiving light emitted from the first plurality of light emitting particles; and
electronics for activating the first plurality of light emitting particles and receiving an output from the detector for determining a pulse rate.

12. The photoplethysmographic sensing system of claim 11 wherein the light emitting device includes a second plurality of light emitting particles, wherein the electronics further determines a blood oxygen level.

13. The photoplethysmographic sensing system of claim 11 wherein the first plurality of light emitting particles comprise a first plurality of quantum dots.

14. The photoplethysmographic sensing system of claim 12 wherein the second plurality of light emitting particles comprise a second plurality of quantum dots.

15. The photoplethysmographic sensing system of claim 11 wherein the flexible material comprises a flexible polymeric substrate.

16. The photoplethysmographic sensing system of claim 11 further comprising one of a light source and a voltage source for activating the light emitting device.

17. The photoplethysmographic sensing system of claim 11 further comprising an accelerometer coupled to the light emitting device for compensating for motion imparted to the light emitting device.

18. The photoplethysmographic sensing system of claim 11 wherein the detector is positioned adjacent the light emitting device.

19. A method for determining a pulse rate of a user by a photoplethysmographic sensing system, comprising:
emitting light having a first wavelength from a plurality of light emitting particles uniformly distributed in a single, flexible emission source at a first time and a second time;
receiving the light after one of passing through, or reflecting from, blood of the user; and
calculating the pulse rate based on the amount of light received at the first and second times.

20. The method of claim 19 wherein the emitting step further comprises emitting light having a second wavelength from a second plurality of light emitting particles.

21. The method of claim 20 wherein the emitting light having a first wavelength comprises emitting light from a first plurality of quantum dots and the emitting light having a second wavelength comprises emitting light from a second plurality of quantum dots.
